(19) United States
(12) Patent Application Publication
(10) Pub. No.: US 2025/0041409 A1
(43) Pub. Date: Feb. 6, 2025

(54) ANTI-PD1 MONOCLONAL ANTIBODY, PHARMACEUTICAL COMPOSITION THEREOF AND USE THEREOF

(71) Applicant: CTTQ-Akeso (Shanghai) Biomed. Tech. Co., Ltd., Shanghai (CN)

(72) Inventors: Baiyong Li, Shanghai (CN); Yu XIA, Shanghai (CN); Zhongmin Maxwell WANG, Shanghai (CN); Peng ZHANG, Shanghai (CN)

(21) Appl. No.: 18/780,086

(22) Filed: Jul. 22, 2024

Related U.S. Application Data
(63) Continuation of application No. 16/327,083, filed on Feb. 21, 2019, now Pat. No. 12,076,398.

(30) Foreign Application Priority Data
Aug. 23, 2016 (CN) 201610705763.5

(51) Int. Cl. A61K 39/395; A61P 35/02; C07K 16/28; G01N 33/577; G01N 33/68

(52) U.S. Cl. CPC A61K 39/395; A61P 35/02; C07K 16/28; G01N 33/577; G01N 33/68

(57) ABSTRACT

An anti-PD1 (programmed cell death 1) monoclonal antibody or an antigen-binding fragment thereof, a pharmaceutical composition thereof and use thereof. The heavy chain variable region of the monoclonal antibody comprises CDRs (complementary determining region) of amino acid sequences as shown in SEQ ID NO:9-11; and/or the light chain variable region of the monoclonal antibody comprises CDRs of amino acid sequences as shown in SEQ ID NO: 12-14. The monoclonal antibody can bind to PD1 specifically, relieve immunosuppression of PD1 on an organism specifically and activate T lymphocytes.

### CROSS-REFERENCE TO RELATED APPLICATIONS

The present application is a continuation of U.S. patent application Ser. No. 16/327,083, filed on Feb. 21, 2019, (now allowed), which is a U.S. National Phase Application, filed under 35 U.S.C. § 371, of International Application No. PCT/CN2017/098465, filed on Aug. 22, 2017, which claims priority to Chinese Patent Application No. 201610705763.5, filed on Aug. 23, 2016, the contents of each of which are incorporated herein by reference in their entireties.

### DESCRIPTION OF THE TEXT FILE SUBMITTED ELECTRONICALLY

The contents of the electronic sequence listing (AKSO_003_02US_SeqList_ST26.xml; Size: 21,806 bytes; and Date of Creation: Jul. 22, 2024) are herein incorporated by reference in its entirety.

### TECHNICAL FIELD

The present invention belongs to the field of tumor therapy and molecular immunology, relating to an anti-PD1 antibody, the pharmaceutical composition and methods of use. Specifically, the present invention relates to an anti-PD1 monoclonal antibody.

### TECHNICAL BACKGROUND

The transmembrane receptor PD1 (programmed cell death 1, also known as PD-1) is a member of the CD28 gene family, expressed in activated T cells, B cells and myeloid cells. Both ligands of PD1 (i.e. PDL1 and PDL2) belong to the B7 superfamily; wherein PDL1 is broadly expressed in a variety of cells including T cells, B cells, endothelial cells and epithelial cells, while PDL2 is only expressed in antigen presenting cells such as dendritic cells and macrophages.

T cells play a very important role in the elimination of viral infections, and T cell antiviral response is usually associated with immunopathogenesis. PD1 plays a vital role in the negative regulation of T cell activation. Although PD1-mediated negative regulation on T cells can reduce tissue damage caused by infection, blocking or inhibiting the negative regulatory effect of PD1 may lead to autoimmune diseases, for example, pancreatic virus infection can be more effectively eliminated in PD1 gene knockout mice, but may lead to more severe liver damage (Isai et al., 2003, J.Exp.Med. 198:39-50). In addition, tumors with highly expressed PD1 often develop into cancers that are difficult to detect (Hamanishi et al., 2007, Proc.Natl.Acad.Sci.USA 104:3360-5). An established method to regulate PD1 expression is through injection of antibodies into the body.

Due to the broad antitumor prospects and astounding efficacy of PD1 antibody, it is generally believed that antibodies against PD1 pathways will lead to breakthroughs in the treatment of a variety of tumors: non-small cell lung cancer, renal cell carcinoma, ovarian cancer, melanoma (Homet M.B., Parisi G., et al., Anti-PD1 Therapy in Melanoma. Semin Oncol. 2015 June; 42(3):466-473), leukemia, and anemia (Held SA, Heine A, et al., Advances in immunotherapy of chronic myeloid leukemia CML. Curr Cancer Drug Targets. 2013 September; 13(7):768-74).

Ever since the revelation of the unprecedented clinical efficacy data at the annual meetings of American Association for Cancer Research (AACR) and American Society of Clinical Oncology (ASCO) in 2012 and 2013, PD1 antibodies have become the hottest new drugs in R&D in the global pharmaceutical industry.

At present, there is still a need to develop new anti-PD1 antibodies with better binding efficiency to effectively block the binding of PD1 to PDL1.

### SUMMARY OF THE INVENTION

Through in-depth research and creative work, by immunizing mice with recombinant PD1 expressed in mammalian cells expression system as antigen, the inventors obtained hybridoma cells via fusion of mouse splenocytes and myeloma cells. By screening a large number of samples, the inventors obtained the hybridoma cell line LT003 (CCTCC Deposit Accession No.: C2015105).

The inventors surprisingly found that the hybridoma cell line LT003 is capable of secreting a specific monoclonal antibody (named 14C12) binding specifically to PD1, and this monoclonal antibody can effectively block the association of PD1 to PDL1.

Furthermore, the inventors generated a humanized anti-PD1 antibody (named 14C12H1L1) in a creative way.

More surprisingly, the inventors found that the antibodies 14C12 and 14C12H1L1 herein can effectively bind to human T cells, activate T cells and induce the secretion of IFN-γ and IL-2 from human lymphocytes. The antibodies 14C12 and 14C12H1L1 herein have the potential to become drugs for preventing and treating malignancies including lung cancer, melanoma, renal cancer, ovarian cancer and leukemia, as well as anemia.

The following are provided by the present invention:

The present invention relates to a monoclonal antibody or its antigen-binding fragments thereof, wherein,
the heavy chain variable region (V_{H}) of the said monoclonal antibody comprises: CDRs with the amino acid sequences of SEQ ID NO:9-11; and/or
the light chain variable region (V_{L}) of the said monoclonal antibody comprises: CDRs with the amino acid sequences of SEQ ID NO: 12-14.

In some examples of the present invention, the said monoclonal antibody or its antigen-binding fragments thereof, wherein,
the amino acid sequence of V_{H} of the monoclonal antibody is chosen from SEQ ID NO:2 and SEQ ID NO:6; and/or
the amino acid sequence of V_{L} of the monoclonal antibody is chosen from SEQ ID NO:4 and SEQ ID NO:8.

In an example of the present invention, the said monoclonal antibody or its antigen-binding fragments thereof, wherein, the monoclonal antibody comprises:
(1) V_{H} shown by SEQ ID NO:2 and V_{L} shown by SEQ ID NO:4;
(2) V_{H} shown by SEQ ID NO:6 and VL shown by SEQ ID NO:8.

The variable regions in heavy and light chains of an antibody govern binding activity. Each chain contains three hypervariable regions, namely, the complementary determining region (CDR) (HCDR1, HCDR2 and HCDR3 in heavy (H) chain, and LCDR1, LCDR2 and LCDR3 in light (L) chain), which are defined by Kabat, et al. (Sequences of Proteins of Immunological Interest, Fifth Edition (1991), volume 1-3, NIH Publication 91-3242, Bethesda MD).

Through techniques well-known to technical personnel in the field described herein, for example, analyzing the amino acid sequences in the CDR of the monoclonal antibody sequences in Items (1) and (2) above through VBASE2 database:

The antibodies 14C12 and 14C12H1L1 herein comprises the same CDR:

Wherein the amino acid sequences of the 3 CDR regions of V_{H} are as follows:

HCDR1:
(SEQ ID NO: 9)
GFAFSSYD,
HCDR2:
(SEQ ID NO: 10)
ISGGGRYT,
HCDR3:
(SEQ ID NO: 11)
ANRYGEAWFAY;

Wherein the amino acid sequences of the 3 CDR regions of V_{L} are as follows:

LCDR1:
(SEQ ID NO: 12)
QDINTY,
LCDR2:
(SEQ ID NO: 13)
RAN,
LCDR3:
(SEQ ID NO: 14)
LQYDEFPLT.

In certain example, the said monoclonal antibody or its antigen-binding fragment thereof, wherein the said monoclonal antibody or its antigen-binding fragment are selected from Fab, Fab′, F(ab′)2, Fd, Fv, dAb, CDRs, single chain antibodies (e.g. scFv), humanized antibodies, chimeric antibodies, or bispecific antibodies.

In certain embodiments, the said monoclonal antibody or its antigen-binding fragment thereof, wherein the said monoclonal antibody binds to PD1 protein with K_{D} less than approximately 10⁻⁵ M, for example, less than approximately 10⁻⁶ M, 10⁻⁷ M, 10⁻⁸ M, 10⁻⁹ M, 10⁻¹⁰ M, or less; preferably, detected by Fortebio molecular interaction equipment.

In certain embodiments, the said monoclonal antibody or its antigen-binding fragment thereof, wherein the said monoclonal antibody binds to PD1 protein with EC₅₀ less than approximately 100 nM, for example, less than 10 nM, 1 nM, 0.9 nM, 0.8 nM, 0.7 nM, 0.6 nM, 0.5 nM, 0.4 nM, 0.3 nM, 0.2 nM, 0.1 nM or less. Specifically, the said EC₅₀ is determined by an indirect ELISA.

In certain embodiments, the said monoclonal antibody or its antigen-binding fragment thereof, wherein the said monoclonal antibody binds to PD1 protein with K_{D} less than approximately 10⁻⁵ M, such as less than approximately 10⁻⁶ M, 10⁻⁷ M, 10⁻⁸ M, 10⁻⁹ M, 10⁻¹⁰ M, or less,

In certain embodiments, the said monoclonal antibody or its antigen-binding fragment thereof, wherein the said monoclonal antibody contains non-CDR regions from species other than mouse, for example, from human.

In certain embodiments, the said monoclonal antibody or its antigen-binding fragments thereof, wherein the said monoclonal antibody is produced by the hybridoma cell line LT003, and the said hybridoma cell line LT003 is preserved in China Center for Type Culture Collection (CCTCC) with the CCTCC Deposit Accession NO: C2015105.

The present invention relates to an isolated nucleic acid molecule comprising a nucleotide sequence capable of encoding V_{H} of the antibody, wherein,
the V_{H} of the said antibody comprises: CDRs with the amino acid sequences from SEQ ID NO: 9-11;

Specifically, the heavy chain of the said antibody has the amino acid sequences from SEQ ID NO: 2 and SEQ ID NO:6;

More specifically, the said nucleic acid molecule has the nucleotide sequences from SEQ ID NO: 1 or SEQ ID NO:5.

The present invention relates to an isolated nucleic acid molecule comprising a nucleotide sequence capable of encoding V_{L} of the antibody, wherein,
the V_{L} of the antibody comprises CDRs with the amino acid sequences from SEQ ID NO:12-14;

Specifically, the V_{L} of the said antibody has the amino acid sequences from SEQ ID NO:4 or SEQ ID NO:8;

More specifically, the said nucleic acid molecule has the nucleotide sequences from SEQ ID NO: 3 or SEQ ID NO:7.

The present invention relates to a vector comprising the isolated nucleic acid molecule described in the present invention.

The present invention relates to a host cell comprising the isolated nucleic acid molecule described in the present invention, or vector described in the present invention.

The present invention relates to a method for preparing the monoclonal antibody or its antigen-binding fragments thereof described in the present invention, by culturing the host cell in the present invention under appropriate conditions, and recovering the said monoclonal antibody or its antigen-binding fragments thereof from the cell culture.

The present invention relates to the hybridoma cell line LT003 that is preserved in the China Center for Typical Culture Collection (CCTCC) with the CCTCC Deposit Accession NO: C2015105.

The present invention relates to a conjugate that consist of monoclonal antibody or its antigen binding fragments and conjugating part, wherein, the said monoclonal antibody is monoclonal antibody or its antigen binding fragments described in the present invention, and the said conjugating part as a detectable marker. Specifically, the said conjugating part are radioactive isotopes, fluorescein, luminescent materials, colorful substances, or enzymes.

The present invention relates to reagent kits, consisting of the monoclonal antibody or its antigen binding fragments, or the conjugates thereof described in the invention;

Specifically, the reagent kits may contain a secondary antibody, which specifically recognizes the said monoclonal antibody or its antigen binding fragments; optionally, the said secondary antibody may contain detectable markers, such as radioactive isotopes, fluorescein, luminescent materials, colorful substances, or enzymes.

The present invention relates to use of the said monoclonal antibody or its antigen binding fragments, or the conjugates thereof described in the invention in preparation of reagent kits, the said reagent kits are used in detection of the existence or the level of PD1 in samples.

The present invention relates to a pharmaceutical composition comprising the said monoclonal antibody or its antigen binding fragments, or the conjugates thereof described in the invention. Optionally, it may also comprise a pharmaceutically acceptable carrier or excipient.

The present invention relates to use of the said monoclonal antibody or its antigen binding fragments or the conjugates thereof described in the invention in preparing drugs for prevention and/or treatment and/or adjuvant treatment and/or diagnosis of tumors or anemia; specifically, the said tumors may be melanoma, renal cancer, prostate cancer, bladder cancer, colorectal cancer, gastrointestinal cancer, liver cancer, non-small cell lung cancer, ovarian cancer and leukemia.

The present inventors have found through animal experiments that, 14C12H1L1 can effectively inhibit the growth of MC38 tumor cells inoculated at right side subcutaneously in PD-1 HuGEMM mice, which the antibody drug 14C12H1L1 can significantly inhibit the tumor growth in PD-1 HuGEMM tumor-bearing mice, having an efficacy equivalent to the marketed monoclonal antibody drug Nivolumab that is approved drug targeting the same target.

The present invention relates to use of the monoclonal antibody or its antigen-binding fragments or the conjugates thereof described in the present invention for preparation drugs with the following purposes:
Blocking the binding of PD1 to PD1 ligand,
Regulating (e.g. Down-regulating) PD1 activity or level,
Relieving the immunosuppression of PD1, or
Up-regulating IFN-γ and/or IL-2 expressions in T lymphocytes;

Specifically, the said PD1 ligand is PDL1 or PDL2, preferably PDL1.

The present invention relates to an in vivo or in vitro method to apply to cells or subjects in need with an effective dose of the monoclonal antibody or its antigen-binding fragments or the conjugates thereof described in the present invention, and the said method is selected from the following:
Methods to block the binding of PD1 to PD1 ligand,
Methods to regulate (e.g. down-regulate) PD1 activity or level,
Methods to relieve the immunosuppression of PD1, or
Methods to up-regulate IFN-γ and/or IL-2 expressions in T lymphocytes;

Specifically, the said PD1 ligand is PDL1 or PDL2, preferably PDL1.

In a specific example of the present invention, the said in vitro method is intended for non-therapeutic or-diagnostic purposes.

Interferon γ (IFNγ), is mainly naturally produced by natural killer (NK) cells and natural killer T (NKT) cells, or produced by effector T cells consisting of CD4 Th1 cells and CD8 cytotoxic T lymphocytes after being stimulated by specific antigens. As an important cytokine of innate and acquired immune, IFNγ plays an import role in antagonizing or inhibiting viral, some bacterial and protozoon infections. In the meantime, IFNγ can activate macrophages and induce the expression of type 2 major histocompatibility complex (MHC) to activate immune responses to control the progression of tumors (Schoenborn JR, Wilson CB. Regulation of Interferon-y During Innate and Adaptive Immune Responses. Advances in Immunology 2007; 96:41-101). In the in vitro study of the present invention, the anti-PD1 antibody can induce the secretion of IFNγ to activate immune responses.

Interleukin 2 (IL-2) produced by T cells is a growth factor regulating T cell subsets and a crucial factor regulating immune responses, promoting activated B cells proliferation, and participating in antibody responses, hematopoiesis and oncological surveillance. Recombinant human IL-2 has been approved by the U. S. FDA for the treatment of malignant tumors (including melanoma, renal tumor, etc.) while undergoing clinical studies for the treatment of chronic viral infections (Chavez, A.R., et al., Pharmacologic administration of interleukin-2. Ann N Y Acad Sci, 2009.1182: p.14-27). In vitro studies, the anti-PD1 antibody of the present invention can specifically relieve the immunosuppression of PD1, activate T cells and induce IL-2 production, displaying promising prospects of extensive applications in gene therapies for neoplastic and parasitic diseases.

The monoclonal antibody or its antigen-binding fragments or the conjugates thereof described in the present invention is used for the prevention and/or treatment and/or adjuvant treatment and/or diagnosis of tumors or anemia; specifically, the said tumors may be melanoma, renal cancer, prostate cancer, bladder cancer, colorectal cancer, gastrointestinal cancer, liver cancer, non-small cell lung cancer, ovarian cancer or leukemia.

The monoclonal antibody or its antigen-binding fragments or the conjugates thereof described in the present invention is used to:
Block the binding of PD1 to PD1 ligand,
Regulate (e.g. down-regulate) PD1 activity or level,
Relieve the immunosuppression of PD1, or
Up-regulate IFN-γ and/or IL-2 expressions in T lymphocytes;

Specifically, the said PD1 ligand is PDL1 or PDL2, preferably PDL1.

In a specific example of the present invention, the monoclonal antibody or its antigen-binding fragments or the conjugates thereof described in the present invention only blocks the binding of PD1 to PDL1.

The present invention relates to a method for the prevention and/or treatment and/or adjuvant treatment and/or diagnosis of tumors or anemia, including the procedure to apply to subjects with an effective dose of the monoclonal antibody or its antigen-binding fragments or the conjugates thereof described in the present invention; specifically, the said tumors may be melanoma, renal cancer, prostate cancer, bladder cancer, colorectal cancer, gastrointestinal cancer, liver cancer, non-small cell lung cancer, ovarian cancer or leukemia.

Unless otherwise defined herein, scientific and technical terms used in connection with the present invention shall have the meanings that are commonly understood by those of ordinary skill in the art. Furthermore, laboratory techniques of cell and tissue culture, molecular genetics, oligo-or polynucleotide chemistry, and immunology described herein are those well-known and commonly used in the art. Meanwhile, to better understand the present invention, the following terms, unless otherwise indicated, shall be understood to have the following meanings:

As used in this invention, the term “Amino acid sequence of PD1 protein (Programmed cell death protein 1, NCBI GenBank: NP_005009.2)” comprises the full length PD1 protein, or PD1ECD (extracellular segment of PD1) or PD1ECD-containing fragments; and also comprises the fusion protein of PD1ECD, for example, fused with the Fc protein fragments of mouse or human IgG (mFc or hFc). Furthermore, understood by those of ordinary skill in the art, the amino acid sequence of PD1 protein can have naturally or artificial mutations (including but not limited to substitutions, deletions, and/or additions), not affecting its biological functions. Therefore, in the present invention, the term “PD1 protein” should include all such sequences and their natural or artificial variants. Furthermore, when describing the sequence fragments of PD1 protein, the said sequence fragments comprise both the sequence fragments and the corresponding sequence fragments in its natural or artificial variants.

As used in this invention, the term “Amino acid sequence of PDL1 protein (Programmed death-ligand 1, NCBI Genebank ID: NP_054862.1)” comprises the full length PDL1 protein, or PDL1ECD (extracellular segment of PDL1) or PDL1ECD-containing fragments; and also comprises the fusion protein of PDL1ECD, for example, fused with the Fc protein fragments of mouse or human IgG (mFc or hFc). Furthermore, understood by those of ordinary skill in the art, the amino acid sequence of PDL1 protein can have naturally or artificial mutations (including but not limited to substitutions, deletions, and/or additions), not affecting its biological functions. Therefore, in the present invention, the term “PDL1 protein” should include all such sequences and their natural or artificial variants. Furthermore, when describing the sequence fragments of PDL1 protein, the said sequence fragments comprise both the sequence fragments and the corresponding sequence fragments in its natural or artificial variants.

As used in this invention, the term “EC₅₀” refers to the concentration for 50% of maximal effect.

As used in this invention, the term “antibody” refers to immunoglobulin proteins, which typically composed of two pairs of polypeptide chains (each pair has a “light” (L) chain and a “heavy” (H) chain). The light chains are classified as κ and λ light chains. The heavy chains are classified as ρ, δ, γ, α, or ε, and respectively, define isotype antibodies as IgM, IgD, IgG, IgA and IgE. In light chains and heavy chains, variable regions and constant regions are connected by a “J” region consisting of about 12 or more amino acids. The heavy chain also contains a “D” region with about 3 or more amino acids. Each heavy chain contains a variable region (V_{H}) and a constant region (C_{H}), which consists of 3 domains (C_{H}1, C_{H}2, and C_{H}3). Each light chain contains a variable region (V_{L}) and a constant region (C_{L}), which consists of one domain C_{L}. The constant region can mediate the binding of immune globulin to host tissues or factors, including various cells in the immune system (e.g., effector cells) and the complement component 1q (C1q) of the classical complement system. V_{H} and V_{L} can also be subdivided into regions with high variability (called complementarity determining region (CDR)), which are separated by relatively conservative regions called framework regions (FR). From the amino terminus to the carboxyl terminus, each V_{H} and V_{L} is composed of 3 CDRs and 4 FRs, in the order of FR1, CDR1, FR2, CDR2, FR3, CDR3, and FR4. The variable regions (V_{H} and V_{L}) of the heavy chain and light chain form the antibody binding site. Distribution of amino acids to the regions or domains follow the definitions by Kabat in Sequences of Proteins of Immunological Interest (National Institutes of Health Bethesda, MD (1987 and 1991)), or Chothia & Lesk (1987) Mol. Biol., 196:901-917; or Chothia et al. (1989) Nature, 342:878-883. The term “antibody” is not restricted by any particular method of producing them. For example, it includes, in particular, recombinant antibodies, monoclonal antibodies, and polyclonal antibodies. Antibodies can be different isotypes, for example, IgG (such as IgG1, IgG2, IgG3, or IgG4 subtype), IgA1, IgA2, IgD, IgE, or IgM antibodies.

As used in this invention, the term “antigen binding fragments” refers to a polypeptide containing fragments of a full-length antibody, maintaining the ability to bind specifically to the same antigen, and/or to compete with the full length antibody to bind to the antigen, which is also called “the antigen binding portion”. See Fundamental Immunology, Ch. 7 (Paul, W., ed. 2, Raven Press, N. Y. (1989)), including the entire article and references in this invention for all purposes. Antigen binding fragments can be generated by recombinant DNA techniques or by cleaving intact antibodies with proteolytic enzymes or chemicals. In some cases, the antigen binding fragments include Fab, Fab′, F(ab′)₂, Fd, Fv, dAb, and CDR fragments, single chain antibodies (e.g., scFv), chimeric antibodies, diabody, and the polypeptides that at least contains an antibody portion which is sufficient to confer a specific antigen binding capacity to the polypeptides.

In some cases, the antigen binding fragment is a diabody, namely, a dimeric antibody fragment, whose V_{H} and V_{L} domains are expressed on a single polypeptide chain, however, because of using a too short linker the to allow pairing between the two domains of the same chain, the domains are forced to pair with complementary domains on another chain to generate two antigen binding sites (see, for example, Holliger P. et al., Proc. Natl. Acad. Sci. USA 90:6444-6448 (1993), and Poljak R. J. et al., Structure 2:1121-1123 (1994)).

Using conventional techniques known by those of ordinary skill in the art (such as recombinant DNA technology or enzymatic/chemical cleavage), an antigen binding fragment (such as the above described antibody fragments) may be obtained from a given antibody (e.g. monoclonal antibodies 14C12, 14C12H1L1 provided herein in the invention), and screened for specificity in the same manner as for the full antibody.

In this invention, unless specified otherwise, the term “antibody” refers to not only the intact antibody, but also the antigen binding fragments of the antibody.

As used in this invention, the terms “mAb” and “monoclonal antibodies” refers to an antibody or a fragment of an antibody that is derived from a group of highly homologous antibodies, i.e. from a group of identical antibody molecules, except for mutations that may arise spontaneously. Monoclonal antibody has high specificity against a single epitope on the antigen. Polyclonal antibodies are different from monoclonal antibodies, containing at least 2 or more different antibodies, which usually recognize different epitopes on the antigen. Monoclonal antibodies can be obtained with hybridoma technology reported originally by Kohler et al., (Nature, 256:495, (1975)), as well as recombinant DNA Technology (see U.S. Pat. No. 4,816,567).

As used in this invention, the term “humanized antibody” refers to an antibody or its fragments, derived from a human immunoglobulin (receptor antibody), whose CDRs or part of CDRs are replaced by the CDR regions of a non-human antibody (donor antibody), where the donor antibody may be a non-human antibody (for example, mice, rats, or rabbits) with predictive specificity, binding affinity, or reactivity. In addition, some amino acid residues of the receptor antibody framework region (FR) can also be replaced by the corresponding amino acid residues of the non-human source, or replaced by the amino acid residues of other antibodies to further improve or optimize the performance of the antibody. For more details on humanized antibodies, see for example Jones, et al., Nature, 321:522-525 (1986); Reichmann et al., Nature, 332:323-329 (1988); Presta, Curr. Op. Struct. Biol., 2:593-596 (1992); and Clark, Immunol. Today, 21:397-402 (2000).

As used in this invention, the term “isolate” or “isolated” means obtained by artificial means in the natural state. If there is a certain kind of “isolated” matter or component in nature, it may be due to the change in its natural environment, or isolated from the natural environment, or both. For example, polynucleotide or polypeptide in a natural existence in a living animal will be called “isolated” if it was separated with high purity in the same natural state. The term “isolate” or “isolated” does not exclude existence of artificial or synthetic material, or other impurities that does not affect the activity.

As used in this invention, the term “vector” refers to a nucleic acid delivery vehicle that can be inserted with polynucleotide. The vector that can have the protein that is encoded by the inserted polynucleotide expressed is called an expression vector. Vectors can be inserted into the host cell by transformation, transduction, or transfection, so the genetic substances carried by the vector can be expressed in the host cell. Vectors are well known to the technical personnel in the field, including but not limited to: plasmid; phasmid; cosmid; artificial chromosome such as yeast artificial chromosome (YAC), bacterial artificial chromosome (BAC), or P1 derived artificial chromosome (PAC); phage such as λ phage or M13 phage and animal viruses etc. Animal viruses may include but not limited to, reverse transcriptase virus (including lentivirus), adenovirus, adeno-associated virus, herpes virus (e. g. herpes simplex virus), chicken pox virus, baculovirus, papilloma virus, and papova virus (such as SV40). A vector can contain multiple components that control expression, including but not limited to, promoter, transcription initiation factor, enhancer, selection element, and reporter gene. In addition, the vector may also contain replication initiation site.

As used in this invention, the term “host cell” refers to cells that can import vectors, including but not limited to, prokaryotic cells such as E. coli and Bacillus subtilis, fungal cells such as yeast and Aspergillus, insect cells such as S2 drosophila cells and Sf9, or animal cells such as fibroblast cells, CHO cells, COS cells, NSO cells, HeLa cells, BHK cells, HEK293 cells or human cells.

As used in this invention, the term “specific binding” refers to a non-random binding between two molecules, such as the interaction between the antibody and its target antigen. In some embodiments, a specific binding of an antibody to an antigen means an affinity (K_{D}), for example less than about 10⁻⁵ M, in particular, less than 10⁻⁶ M, 10⁻⁷ M, 10⁻⁸ M, 10⁻⁹ M, 10⁻¹⁰ M, or less.

As used in this invention, the term “K_{D}” refers to the dissociation equilibrium constant of specific interaction between antibody and antigen, to describe the binding affinity between antibodies and antigens. The smaller the equilibrium dissociation constant is, the tighter the antibody binds antigen, the higher the affinity between the antibody and the antigen is. Typically, antibodies (e.g., monoclonal antibodies 14C12, 14C12H1L1 in the present invention) bind to antigens (e.g., PD1 protein) with a K_{D} less than approximately 10⁻⁵ M, for example, less than 10⁻⁶ M, 10⁻⁷ M, 10⁻⁸ M, 10⁻⁹ M or 10⁻¹⁰ M or even less. K_{D} can be measured by any method well known to the technical personnel in the field, for example, using Fortebio Octet System As used in this invention, the terms “monoclonal antibody” and “mAb” have the same meaning and are used interchangeably; the terms “polyclonal antibody” and “PcAb” have the same meaning and are used interchangeably; the terms “polypeptide” and “protein” have the same meaning and are used interchangeably. Also in the present invention, amino acids are usually represented by single letter or three letter abbreviations known to this field. For example, Alanine can be represented as A or Ala.

As used in this invention, the terms “hybridoma” and “hybridoma cell line” can be used interchangeably, and when mentioned herein, include the subclone and progeny cells of hybridoma. For example, when mentioned herein, hybridoma cell line LT003 also includes the subclone and progeny cells of LT003.

As used in this invention, the term “pharmaceutically acceptable carrier or excipient” refers to a carrier and/or an excipient pharmaceutically and/or physiologically compatible with subjects and active ingredients, and widely recognized in the field herein (Remington's Pharmaceutical Sciences. Edited by Gennaro AR, 19th ed. Pennsylvania: Mack Publishing Company, 1995), including but not limited to: pH adjustors, surfactants, adjuvants, and ionic strength enhancers. For example, pH adjustors include but not limited to phosphate buffer solution; surfactants include but not limited to cationic, anionic or nonionic surfactants such as Tween-80; ionic strength enhancers include but not limited to sodium chloride.

As used in this invention, the term “effective dose” is defined as an amount of a therapeutic sufficient to achieve or at least partially achieve the desired effect. For example, effective prevention dose (e.g. for cancer) is the amount to prevent, stop, or delay the occurrence of diseases (e.g. cancer); effective treatment dose is the amount to cure, or at least partially stop, the disease and its complications in patients with the disease. Determination of such an effective dose is entirely within the scope of the capabilities of the technical personnel in the field. For example, the effective treatment dose will depend on the severity of the disease, the overall state of the patient's immune system, the general background of patients such as age, weight and gender, administration method of the said drug, and other concomitant treatments, etc.

### Effects of the Invention

The monoclonal antibodies in the present invention, especially 14C12H1L1, is capable of binding to PD1 specifically, effectively blocking the binding of PD1 to PDL1, and relieving the immunosuppression of PD1 to activate T lymphocytes. Wherein, the PD1 antibody 14C12H1L1 can induce the secretions of IFN-γ and IL-2 much better than the control antibody 5C4 (5C4: PD1 antibody from Medarex Inc.: Alan J.Korman, et al., Human monoclonal antibodies to programmed death 1 (PD1) and methods for treating cancer using anti-PD1 antibodies alone or in combination with other immunotherapeutics, U.S. Pat. No. 8,008,449 B2). The monoclonal antibodies of the present invention, especially 14C12H1L1, have an antitumor effect equivalent to the approved drug Nivolumab for the same target. The antibodies of the present invention have the potential to become or to be prepared into drugs for the prevention and/or treatment of non-small cell lung cancer, renal cell carcinoma, ovarian cancer, melanoma, leukemia or anemia.

### DESCRIPTION OF FIGURES

FIG. 1: SDS-PAGE Results of Humanized Monoclonal Antibody 14C12H1L1. From left to right: 1. 1 μg antibody in non-reduced loading buffer; 2. 1 μg antibody in reduced loading buffer; 5 μL Marker; 3. 1 μg BSA.

FIG. 2: Binding kinetics of Antibody 14C12.

FIG. 3: Binding kinetics of Antibody 14C12H1L1.

FIG. 4: Binding kinetics of Antibody 5C4.

FIG. 5: ELISA results of 14C12H1L1 and 5C4 binding to PD1.

FIG. 6: Competition ELISA results of 14C12H1L1 and 5C4 binding to PD1 against PDL1.

FIG. 7: EC₅₀ of 14C12H1L1 binding to PD1 on the Surface of 293T-PD1 Cells.

FIG. 8: Binding activity of 14C12H1L1 to T Cell Surface Antigen PD1.

FIG. 9: Effect of 14C12H1L1 on IFN-γ Secretion of Mixed Lymphocytes.

FIG. 10: Effect of 14C12H1L1 on IL-2 Secretion of Mixed Lymphocytes.

FIG. 11: Effect of 14C12H1L1 on the Secretion of Cytokine IL-2 Induced by Mixing PBMC, MDA-MB-231 and Raji Cells.

FIG. 12: Effect of 14C12H1L1 on the Tumor Growth of MC38 Tumor Model in PD-1 HuGEMM Mice.

Hybridoma cell line LT003, has been preserved in the China Center for Typical Culture Collection (CCTCC) in Wuhan University, Wuhan, China 430072 on Jun. 16, 2015 with CCTCC Deposit Accession NO: C2015105

### DETAILED DESCRIPTION OF THE INVENTION

The invention will now be described in detail. As will be appreciated by one skilled in the art, the following examples are only used for the description of the invention, and not to be deemed to limit the scope of the invention. The cases without the specific descriptions of techniques or conditions were carried out in accordance with the literature in the field (e.g., Guide to Molecular Cloning, written by J Sambrook, et al, translated by Peitang Huang, et al, third Edition, Science Press) or in accordance with the product instruction manual. The reagents or instruments with no specified manufacturer were all conventional products available commercially.

In the examples below, T cells used were from Akeso Biopharma, Inc.; the BALB/C mice were purchased from Guangdong Medical Laboratory Animal Center. The PD-1 HuGEMM mice used were from Nanjing Galaxy Biopharma Co., Ltd.; MC38 cells were from Shanghai Fudan IBS Cell Center; the marketed drug for the same target, Nivolumab (Opdivo®) used was from Bristol-Myers Squibb Company.

### EXAMPLE 1

### Acquisition of Hybridoma Cell Line LT003 and Preparation of Monoclonal Antibody 14C12

### 1. Establishment of Hybridoma Cell Line LT003

Used PD1-mFc (PD1: Programmed cell death protein 1, NCBI GenBank ID: NP_005009.2) fusion protein as the antigen, and fused the splenocytes of immunized BALB/C mice (purchased from Guangdong Medical Laboratory Animal Center) and mouse myeloma cells into hybridoma cells by currently established method (for example, Stewart, S.J., “Monoclonal Antibody Production”, in Basic Methods in antibody Production and Characterization, Eds.G.C. Howard and D.R. Bethell, Boca Raton: CRC Press, 2000).

Coating the microplate with PD1-mFc as the antigen, and the hybridoma cells were screened by indirect ELISA to obtain hybridoma cells that secrets new antibodies specifically binding to PD1.

Screened out the hybridoma cell lines capable of secreting monoclonal antibodies binding to PD1 by competition ELISA against the ligand PDL1-hFc fused protein (PDL1: Programmed death-ligand 1, NCBI Genebank ID: NP_054862.1), and obtained stable hybridoma cell lines by limited dilution method, and then obtained stable LT003 cell lines by limited dilution method (the monoclonal antibody secreted from LT003 is named 14C12).

Hybridoma cell line LT003 (PD1-14C12), has been preserved in the China Center for Typical Culture Collection (CCTCC) in Wuhan University, Wuhan, China 430072 on Jun. 16, 2015 with CCTCC Deposit Accession NO: C2015105.

### 2. Preparation of Monoclonal Antibody 14C12

LT003 cell line in the present invention was cultured using IMDM medium containing 10% low IgG fetal bovine serum for 7 days, and then the cell culture supernatant was harvested and purified to get the antibody 14C12.

### EXAMPLE 2

### Acquisition of Light-Chain and Heavy-Chain Sequences of Monoclonal Antibody 14C12

Extracted mRNA from the hybridoma cell line LT003 prepared in Example 1 above according to the manual of the cell/bacterial total RNA extraction reagent kit (Tiangen, Product No DP430).

Synthesized cDNA according to the manual of Invitrogen SuperScript® III First-Strand Synthesis System for RT-PCR Kit and carried out PCR amplification.

Directly carried out TA cloning using the PCR amplification products according to the instructions of pEASY-T1 Cloning Kit (Transgen CT101).

Sequenced the TA cloning products, and obtained the following results:

DNA sequencing results of V_{H}: (354 bp)

(SEQ ID NO: 1)
GAGGTCAAACTGGTGGAGAGCGGCGGCGGGCTGGTGAAGCCCGGCGGG
TCACTGAAACTGAGCTGCGCCGCTTCCGGCTTCGCCTTTAGCTCCTAC
GACATGTCATGGGTGAGGCAGACCCCTGAGAAGCGCCTGGAATGGGTC
GCTACTATCAGCGGAGGCGGGCGATACACCTACTATCCTGACTCTGTC
AAAGGGAGATTCACAATTAGTCGGGATAACGCCAGAAATACTCTGTAT
CTGCAGATGTCTAGTCTGCGGTCCGAGGATACAGCTCTGTACTATTGT
GCAAACCGGTACGGCGAAGCATGGTTTGCCTATTGGGGACAGGGCACC
CTGGTGACAGTCTCTGCC

Encoded protein sequence: (118 aa)

(SEQ ID NO: 2)
EVKLVESGGGLVKPGGSLKLSCAASGFAFSSYDMSWVRQTPEKRLEWV
ATISGGGRYTYYPDSVKGRFTISRDNARNTLYLQMSSLRSEDTALYYC
ANRYGEAWFAYWGQGTLVTVSA

DNA sequencing results of V_{L}: (318 bp)

(SEQ ID NO: 3)
GACATTAAGATGACACAGTCCCCTTCCTCAATGTACGCTAGCCTGGGC
GAGCGAGTGACCTTCACATGCAAAGCATCCCAGGACATCAACACATAC
CTGTCTTGGTTTCAGCAGAAGCCAGGCAAAAGCCCCAAGACCCTGATC
TACCGGGCCAATAGACTGGTGGACGGGGTCCCCAGCAGATTCTCCGGA
TCTGGCAGTGGGCAGGATTACTCCCTGACCATCAGCTCCCTGGAGTAT
GAAGACATGGGCATCTACTATTGCCTGCAGTATGATGAGTTCCCTCTG
ACCTTTGGAGCAGGCACAAAACTGGAACTG

Encoded protein sequence: (106 aa)

(SEQ ID NO: 4)
DIKMTQSPSSMYASLGERVTFTCKASQDINTYLSWFQQKPGKSPKTLI
YRANRLVDGVPSRFSGSGSGQDYSLTISSLEYEDMGIYYCLQYDEFPL
TFGAGTKLEL

### 2. Preparation of Recombinant Monoclonal Antibody 14C12 (Re)

Separately cloned the heavy-chain cDNA sequence (the variable region sequence is SEQ ID NO: 1) and the light-chain cDNA sequence (the variable region sequence is SEQ ID NO:3) of 14C12 (Re) into the pUC57simple (provided by GenScript Biotech Corp.) vector (enzymatic cleavage sites: XbaI & BamHI), and obtained pUC57simple-14C12H and pUC57simple-14C12L plasmids, respectively.

Enzymatically cleaved (HindIII & EcoRI) the plasmids pUC57simple-14C12H and pUC57simple-14C12L, respectively, and then sub-cloned the heavy and light chains recovered from electrophoresis into the pcDNA3.1 vector, extracted both recombinant plasmids and co-transfect 293F cells.

After 7 days cell culture, the cell culture supernatant was centrifuged by high-speed centrifugation and filtered by vacuum filtration with microporous membrane and loaded onto the HiTrap MabSelectSuRe column, and then the antibody was eluted with Elution Buffer in one step, and then went through HiTrap Desalting column, and recovered into PBS buffer, and then the recombinant antibody 14C12 (Re) was obtained after further purification.

As validated by ELISA binding activity assay, the recombinant antibody 14C12 (Re) had a binding activity equivalent to the antibody 14C12, and hence can be further used in the subsequent antibody humanization design.

### EXAMPLE 3

### Design of Heavy-Chain and Light-Chain Sequences of Humanized Antibody 14C12H1L1

### 1. Design of Light-chain and Heavy-chain Sequences of Humanized Antibody 14C12H1L1

According to the three-dimensional crystal structure of PD1 protein (Shinohara T, et al., Structure and chromosomal localization of the human PD1 gene (PDCD1). Genomics 1995, 23 (3):704-6)) and the sequences of the antibody 14C12 obtained in Example 2, through computer antibody modeling, amino acids mutation was designed according to the model, and obtained the variable region sequences of the antibody 14C12H1L1 (Heavy chain constant region is Ig gamma-1 chain C region, ACCESSION:P01857 (SEQ ID NO: 15); light chain constant region is Ig kappa chain C region, ACCESSION:P01834 (SEQ ID NO: 16)), as follows:

DNA sequence of heavy chain variable region: (354 bp)

(SEQ ID NO: 5)
GAAGTGCAGCTGGTCGAGTCTGGGGGAGGGCTGGTGCAGCCCGGCGGG
TCACTGCGACTGAGCTGCGCAGCTTCCGGATTCGCCTTTAGCTCCTAC
GACATGTCCTGGGTGCGACAGGCACCAGGAAAGGGACTGGATTGGGTC
GCTACTATCTCAGGAGGCGGGAGATACACCTACTATCCTGACAGCGTC
AAGGGCCGGTTCACAATCTCTAGAGATAACAGTAAGAACAATCTGTAT
CTGCAGATGAACAGCCTGAGGGCTGAGGACACCGCACTGTACTATTGT
GCCAACCGCTACGGGGAAGCATGGTTTGCCTATTGGGGGCAGGGAACC
CTGGTGACAGTCTCTAGT

Encoded protein sequence: (118 aa)

(SEQ ID NO: 6)
EVQLVESGGGLVQPGGSLRLSCAASGFAFSSYDMSWVRQAPGKGLDWV
ATISGGGRYTYYPDSVKGRFTISRDNSKNNLYLQMNSLRAEDTALYYC
ANRYGEAWFAYWGQGTLVTVSS

DNA sequence of light chain variable region: (321 bp)

(SEQ ID NO: 7)
GACATTCAGATGACTCAGAGCCCCTCCTCCATGTCCGCCTCTGTGGGC
GACAGGGTCACCTTCACATGCCGCGCTAGTCAGGATATCAACACCTAC
CTGAGCTGGTTTCAGCAGAAGCCAGGGAAAAGCCCCAAGACACTGATC
TACCGGGCTAATAGACTGGTGTCTGGAGTCCCAAGTCGGTTCAGTGGC
TCAGGGAGCGGACAGGACTACACTCTGACCATCAGCTCCCTGCAGCCT
GAGGACATGGCAACCTACTATTGCCTGCAGTATGATGAGTTCCCACTG
ACCTTTGGCGCCGGGACAAAACTGGAGCTGAAG

Encoded protein sequence: (107 aa)

(SEQ ID NO: 8)
DIQMTQSPSSMSASVGDRVTFTCRASQDINTYLSWFQQKPGKSPKTLI
YRANRLVSGVPSRFSGSGSGQDYTLTISSLQPEDMATYYCLQYDEFPL
TFGAGTKLELK

### EXAMPLE 4

### Preparation and SDS-PAGE Electrophoresis of Humanized Monoclonal Antibody 14C12H1L1

Separately cloned the heavy-chain cDNA (the variable region sequence is SEQ ID NO:5, heavy chain constant region is Ig gamma-1 chain C region, ACCESSION:P01857 (SEQ ID NO: 15)) and light-chain cDNA (the variable region sequence is SEQ ID NO:7, light chain constant region is Ig kappa chain C region, ACCESSION:P01834 (SEQ ID NO:16)) of 14C12H1L1 into the pUC57simple (provided by GenScript Biotech Corp.) vector (enzymatic cleavage sites: XbaI & BamHI) to obtain pUC57simple-14C12H1 and pUC57simple-14C12L1 plasmids, respectively. Sub-cloned thees plasmids into pcDNA3.1 vector respectively (enzymatic cleavage sites: XbaI & BamHI), extracted both recombinant plasmids and co-transfect 293F cells.

After 7 days cell culture, the cell culture was centrifuged by high-speed centrifugation and filtered via vacuum filtration with microporous membrane and loaded onto the HiTrap MabSelectSuRe column, and then the antibody was eluted with Elution Buffer in one step, went through HiTrap Desalting column and recovered into PBS buffer, and purified further to obtain the recombinant antibody 14C12H1L1, which was analyzed by SDS-PAGE electrophoresis.

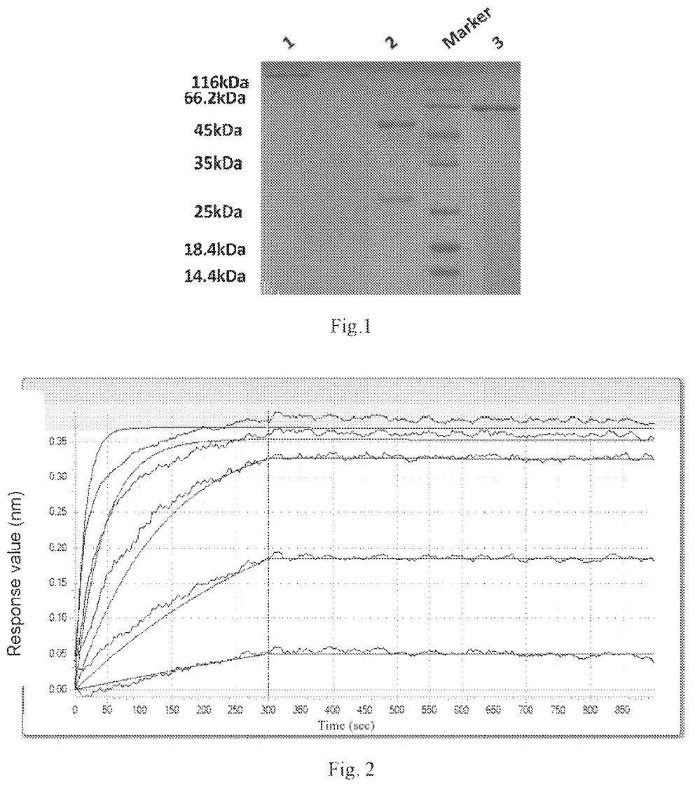

The results were shown in FIG. 1, the reduced target protein appeared at approximately 24.5 kD and 49 KD, and the non-reduced target protein appeared at approximately 147 kD.

### EXAMPLE 5

### Kinetics Measurements of the Antibodies

The binding kinetics of the antibody 14C12 and humanized antibody 14C12H1L1 to the antigen PD1 was measured by Fortebio Octet System.
1. Cleaved PD1-mFc protein with TEV protease, and obtained PD1 antigen by column purification.
2. The antigen PD1 (the concentration at 1 μg/ml) labeled with biotin was immobilized on the surface of SA sensor and equilibrated in PBST buffer, and then bound antibodies 14C12, 14C12H1L1 and 5C4, and the antibodies were diluted with each dilution three-fold relative to the previous one since 200 nM. The dissociation of antigen and antibody were also in PBST.

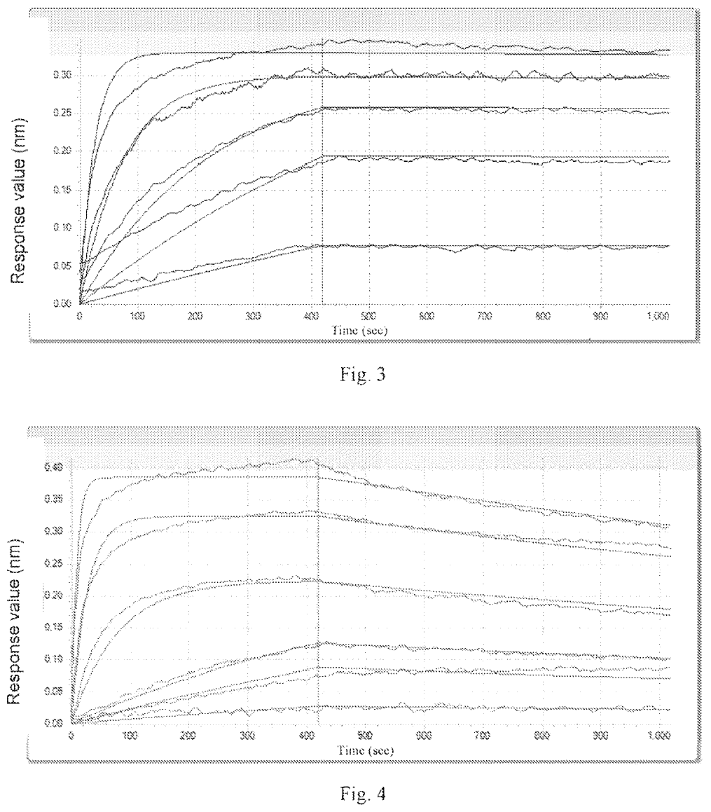

Binding kinetics of the antibodies 14C12, 14C12H1L1 and 5C4 were shown in Table 1 and FIGS. 2, 3 and 4, respectively. The results showed that both 14C12 and 14C12H1L1 had good affinities to PD1, with a higher affinity than that of 5C4.

TABLE 1
Dynamic Parameters of Antibody 14C12.

| Antibody | K_{D} | Kon | Ko | Kdis | Kdis |
|---|---|---|---|---|---|
| Name | (M) | (1/Ms) | Error | (1/s) | Error |
| 14C12 | 1.81E−11 | 3.38E+05 | 8.23E+03 | 6.12E−06 | 1.04E−05 |
| 14C12H1L1 | 2.42E−11 | 3.17E+05 | 5.90E+03 | 7.66E−06 | 8.70E−06 |
| 5C4 | 6.46E−10 | 5.63E+05 | 1.38E+04 | 3.63E−04 | 9.77E−06 |

K_{D}: Dissociation constant; Kₒₙ: Binding rate of antigen and antibody; K_{dis}: Dissociation rate of antigen and antibody; K_{D} = Kdis/Kon.

### EXAMPLE 6

### Binding Activity of Antibody and Antigen PD1 Measured by Indirect ELISA

The binding activities of antibodies 14C12H1L1 and 5C4 to PD1 were measured separately by indirect ELISA as follows:

After incubated with PD1-mFc at 4° C. overnight, the microplate was blocked with 1% BSA at 37° C. for 2 h, and then the antibodies were added separately and incubated at 37° C. for 30 min, and then HRP-labeled secondary antibody (goat anti-human IgG (H+L)) (Jackson, 109-035-088) was added. and then TMB (Neogen, 308177) was added to react for 5 mins and the absorbance was read at the wavelength of 450 nm in a microplate reader.

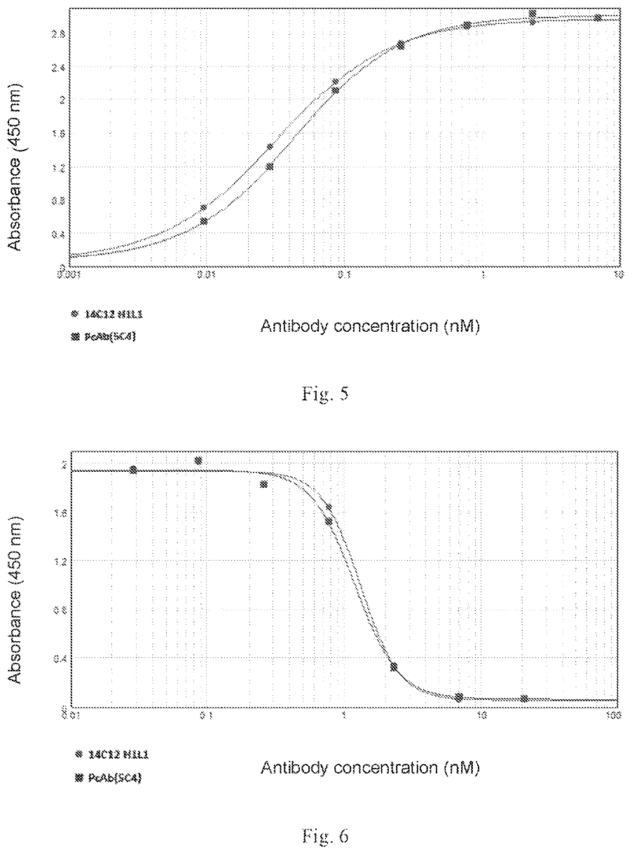

The binding results of antibodies 14C12H1L1 and 5C4 to the antigen PD1 were shown in FIG. 5. As shown in FIG. 5, both the antibodies 14C12H1L1 and 5C4 can bind to PD1 protein effectively with dose-dependency. The absorbance intensities at different doses were shown in Table 2. Through Curve Simulation using quantitative analyses of absorbance values, EC50 of 14C12H1L1 and 5C4 binding with PD1 were then determined to be 0.032 nM and 0.043 nM, respectively.

TABLE 2
Absorbance Intensities of 14C12H1L1 and 5C4 binding to PD1

| Antibody | Antigen coating: | | | |
|---|---|---|---|---|
| concentration | PD1-mFc (0.5 μg/mL) | | | |
| (μg/mL) | 14C12H1L1 | | 5C4 | |
| 1 | 2.970 | 2.954 | 2.959 | 2.991 |
| 0.3 | 2.886 | 2.961 | 2.978 | 3.079 |
| 0.1 | 2.864 | 2.868 | 2.838 | 2.926 |
| 0.03 | 2.674 | 2.669 | 2.617 | 2.659 |
| 0.01 | 2.222 | 2.201 | 1.981 | 2.221 |
| 0.003 | 1.383 | 1.464 | 1.169 | 1.222 |
| 0.001 | 0.676 | 0.736 | 0.527 | 0.548 |
| 0 | 0.062 | 0.062 | 0.065 | 0.073 |

| | |
|---|---|
| Secondary | HRP-labeled secondary antibody |
| Antibody | (goat anti-human IgG) |

### EXAMPLE 7

### Binding Activities of Antibodies to Antigen PD1 against PDL1 by competition ELISA

Binding activities of humanized antibody 14C12H1L1 and 5C4 to antigen PD1 against PDL1 were measured by competition ELISA as follows:

After incubated with PD1-hFc at 4° C. overnight, the microplate was blocked with 1% BSA for 2 h, and antibodies 14C12H1L1 and 5C4 with different concentrations (see Table 3 for the dilute strengths) were mixd with PDL1-mFc for 10 min, and the mixtures were incubated at 37° C. for 30 min, and then corresponding anti-human and anti-mouse enzyme-labeled second antibodies respectively and incubate at 37° C. for 30 min were added. The absorbance at the wavelength of 450 nm was read in a microplate reader.

The binding results of antibodies 14C12H1L1 and 5C4 to the antigen PD1 were shown in FIG. 6. As shown in FIG. 6, the antibody 14C12H1L1 can compete against PDL1 and bind to PD1 protein effectively with dose-dependency. The absorbance intensities at different doses were shown in Table 3. Through Curve Simulation using quantitative analyses of absorbance values, EC50 of 14C12H1L1 and 5C4 binding activity were then determined to be 1.322 nM and 1.199 nM, respectively.

TABLE 3
Competitive Binding of 14C12H1L1 and 5C4 to PD1 against PDL1

| Antibody | Antigen coating | | | |
|---|---|---|---|---|
| concentration/ | PD1-mFc 0.5 μg/mL | | | |
| dilution gradient | 14C12H1L1 | | 5C4 | |
| 1.5 μg/ml | 0.062 | 0.064 | 0.070 | 0.075 |
| 1:3 | 0.069 | 0.064 | 0.081 | 0.086 |
| 1:9 | 0.363 | 0.305 | 0.372 | 0.269 |
| 1:27 | 1.727 | 1.543 | 1.429 | 1.604 |
| 1:81 | 1.892 | 1.752 | 1.766 | 1.881 |
| 1:243 | 1.984 | 2.029 | 2.045 | 2.005 |
| 1:729 | 1.937 | 1.978 | 1.934 | 1.954 |
| 0 | 1.870 | 1.977 | 1.933 | 1.977 |

| | |
|---|---|
| Ligand | PDL1-mFc 0.3 μg/ml |
| Secondary Antibody | HRP-labeled goat anti-mouse secondary antibody |

### EXAMPLE 8

### Binding Activity of Antibodies to Cell Surface Antigen PD1 by Flow Cytometry

Host cells 293T expressing PD1 antigen were constructed, and labeled with the humanized antibody 14C12H1L1 prepared in the present invention (see Example 4). The ability of the antibody 14C12H1L1 to bind specifically to cell surface antigen in its native conformation was analyzed and validated by flow cytometry.

### 1. Construction of Host Cell 293T Expressing PD1 Antigen

The specific steps were as follows:

Construction of the host cell 293T expressing PD1 antigen: 293T cells were transfected with the PD1-containing vector pLenti6.3-PD1 (vector pLenti6.3 was purchased from Invitrogen Corporation) according to the manual of LipofectaminTransfection Kit (purchased from Invitrogen Corporation) to obtain the stable pool of 293T-PD1 expressing PD1 by screening.

### 2. Binding of Antibodies to 293T-PD1 Cell Surface Antigen

Antibody labeling and flow cytometry: The 293T-PD1 obtained by the step above was digested by trypsin, and distributed into tubes each containing 2×10⁵ cells. PD1 antibodies was diluted using PBSbuffer (1% BSA) at concentrations of 50 nM, 10 nM, 5 nM, 1 nM, 0.1 nM and 0.01 nM and added into the tubes and incubated on ice with PD1-expressing 293T cells for 2 h. 100 μL of FITC-labeled goat anti-human secondary antibody (1:500) was added into each tube and incubated on ice for 1 h. After washed with PBS 3 times, cells were re-suspended in 300 μL of PBS and fluorescence signals were measured on the flow cytometer using the FITC channel.

The binding results of the humanized antibody 14C12H1L1 to 293T-PD1 cells were shown in FIG. 7. As shown in FIG. 7, the antibody 14C12H1L1 can bind target PD1 protein expressing on the surface of host cells 293T-PD1 effectively with dose-dependency. The fluorescence intensities at different doses were shown in Table 4. Through Curve Simulation using quantitative analyses of fluorescence intensities, EC50 of 14C12H1L1 binding with PD1 was then determined to be 1.89 nM.

TABLE 4
The Fluorescence Intensities of 14C12H1L1 Binding to
293T-PD1 Surface Antigen Detected by Flow Cytometry

| Concentration (nM) | 0.01 | 0.1 | 1 | 5 | 10 | 50 |
|---|---|---|---|---|---|---|
| Fluorescence | 8.32 | 20.31 | 174.62 | 579.41 | 686.49 | 669.54 |
| Intensity | | | | | | |

### EXAMPLE 9

### Binding Activity of Antibody to T Cell Surface Antigen PD1 by Flow Cytometry

PBMC was isolated by Ficoll-Paque Plus (GE Healthcare LOT No.: 171440-02), further isolated to get CD4⁺ cells, and cells were stimulated with PHA (Shanghai Shenqi Biotech Co., Ltd, 50 μl/ml) for three days, and then washed once with PBS, and antibodies at different concentrations were added and incubated on ice for 1.5 h. The cells were then washed with PBS once after incubation, and the FITC-labeled goat anti-human secondary antibody (Jackson immunoresearch lot. 102155) was added and incubated on ice in the dark for 1 h, and after washed with PBS once, the fluorescence signals were measured on the flow cytometer.

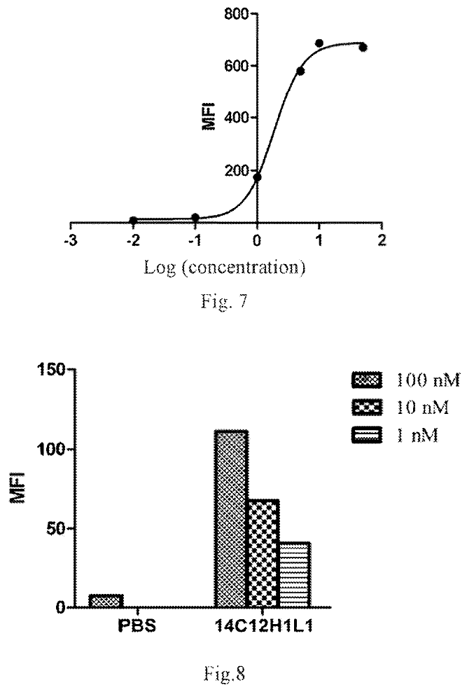

The binding results of humanized antibody 14C12H1L1 to Tcells were shown in FIG. 8. Evidently, the antibody 14C12H1L1 can bind PD1 protein on T cell surface effectively with dose-dependency.

### EXAMPLE 10

### Mixed Lymphocyte Reaction: Secretion of Cytokines IFN-γ, IL-2

PBMC was isolated by Ficoll-Paque Plus (GE Healthcare LOT No.: 171440-02), add the isolated PBMC was induced with IL-4 (Peprotech K2513, 1,000 U/ml) and GM-CSF (Peprotech H1513, 1,000 U/ml) for 6 days, and then TNF-α (Peprotech G1513, 200 U/ml) was added to induce for 3 days to obtain DC cells.

T cells were isolated from PBMC and mixed with the DC cells obtained above in the ratio of 10:1 to culture together with the antibodies 14C12H1L1, 5C4 and hIgG (hIgG as an isotype control) in different ratios for 5-6 days. The secretions of IFN-γ and IL-2 were measured with corresponding ELISA reagent kits (both purchased from Dakewe) respectively.

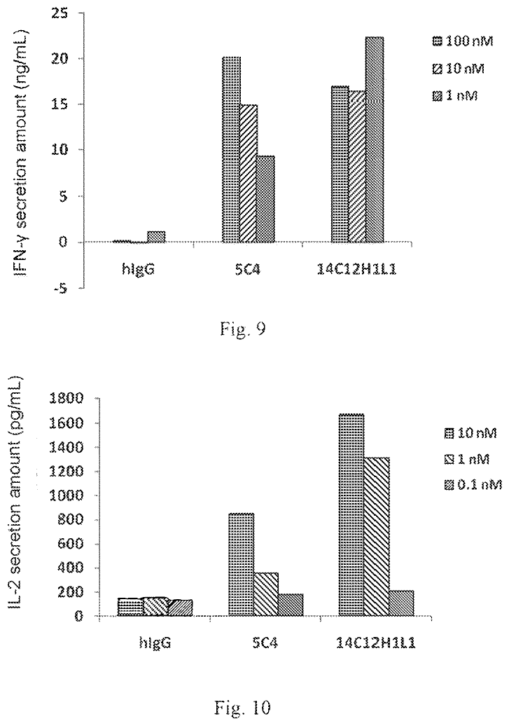

The secretions of IFN-γ and IL-2 after mixed culture of DC cells and T cells were shown in FIG. 9 and FIG. 10. 14C12H1L1 antibody can effectively induce the secretions of IFN-γ and IL-2 with dose-dependency. Antibody 14C12H1L1 can induce higher secretions of both IFN-γ and IL-2 than the control antibody 5C4.

### EXAMPLE 11

### Induced IL-2 Secretion

The isolated PBMC (the same method as in Example 10) was stimulated with PHA (Shanghai Shenqi Biotech Co., Ltd, 50 μl/ml) for 3 days, and then mature PBMC (5×10⁴ cells/well) mixed with Raji cells (Chinese Academy of Sciences Shanghai Branch) (5×10⁴ cells/well) and MDA-MB-231 cells (ATCC) (1×10⁴ cells/well) in a 96-well plate. 100 nM of 14C12H1L1 or control antibody 5C4 or hIgG (hIgG as the isotype control) were added and mixed and cultured together for three days. The secretion of IL-2 was detected with ELISA reagent kit (purchased from Dakewe) according to the instructions of the kit.

The results of IL-2 secretion after mixed cell culture were shown in FIG. 11. As shown in FIG. 11, antibody can effectively induce PBMC to secret IL-2, and the IL-2 secretion induced by 14C12H1L1 is significantly higher than that of control antibody 5C4.

### EXAMPLE 12

### Impact of Antibody 14C12H1L1 on the Tumor Growth of MC38 Tumor Model in PD-1 HuGEMM Mice

MC38 tumor cells (1×10⁶ cells/mouse) were inoculated subcutaneously on the right side of PD-1 HuGEMM mice (human PD-1 transgenic mice). When the mean tumor volume reached approximately 118 mm³, the mice were randomly divided into 4 experimental groups with 8 mice in each group. Antibodies were given through abdominal administration. The specific grouping and dosages were as follows:
Isotype Control group (dose: 8 mg/kg),
14C12H1L1 high-dose group (dose: 8 mg/kg),
14C12H1L1 low-dose group (dose: 0.8 mg/kg),
Nivolumab group (dose: 8 mg/kg).

The above 4 groups were injected with antibodies twice weekly, 5 doses in total. After injection, the tumor sizes were measured twice weekly.

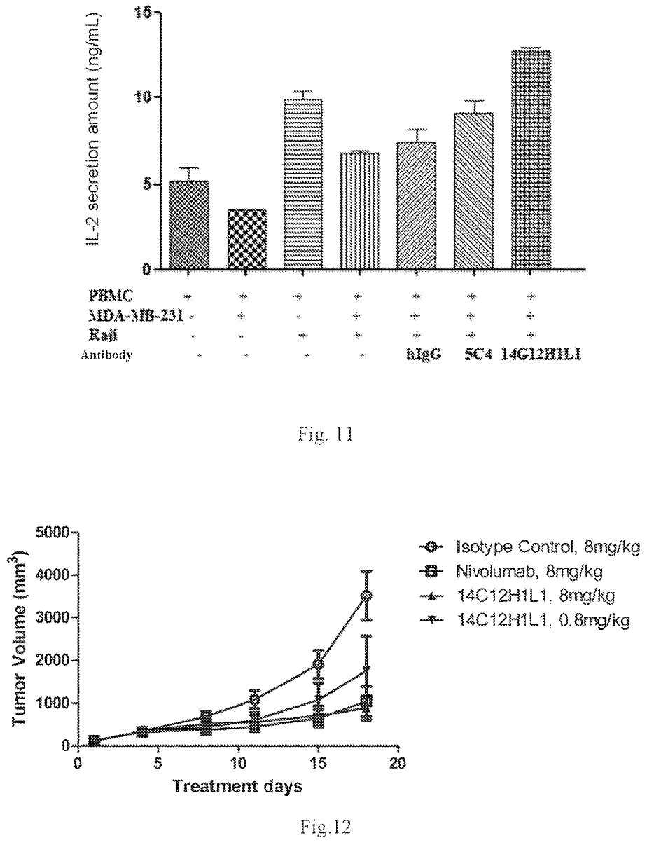

The results were presented in FIG. 12.

The results indicating that:

The tumor sizes in the Nivolumab, 14C12H1L1 high-dose, and 14C12H1L1 low-dose groups were all significantly smaller than those in the Isotype control group statistically (P<0.01, P<0.01, P<0.05, respectively). 14C12H1L1 high-dose group (8 mg/kg) showed a statistically significant antitumor effect on the MC38 tumor model in the PD-1 HuGEMM mice, and had an efficacy equivalent to the approved drug for the same target, Nivolumab (8 mg/kg).

Although specific embodiments of the present invention have been described in detail, as will be appreciated by one skilled in the art, these details may incur various modifications and substitutions according to all the teachings we have disclosed. These changes are all covered by the scope of the present invention. The full scope of the present invention is given by the appended claims and any equivalents.

1.-24. (canceled)

25. A monospecific anti-PD1 antibody, or an antigen-binding fragment thereof, comprising:
a) three heavy chain variable region (V_{H}) complementarity determining regions (HCDRs): HCDR1 having the amino acid sequence set forth in SEQ ID NO: 9, HCDR2 having the amino acid sequence set forth in SEQ ID NO: 10, and HCDR3 having the amino acid sequence set forth in SEQ ID NO: 11; and
b) three light chain variable region (V_{L}) complementarity determining regions (LCDRs): LCDR1 having the amino acid sequence set forth in SEQ ID NO: 12, LCDR2 having the amino acid sequence set forth in SEQ ID NO: 13, and LCDR3 having the amino acid sequence set forth in SEQ ID NO: 14.

26. The monospecific anti-PD1 antibody or antigen-binding fragment thereof of claim 25, comprising:
the V_{H} sequence set forth in SEQ ID NO: 2 or SEQ ID NO: 6; and/or
the V_{L} sequence set forth in SEQ ID NO: 4 or SEQ ID NO: 8.

27. The monospecific anti-PD1 antibody or antigen-binding fragment thereof of claim 26, comprising:
the V_{H} sequence set forth in SEQ ID NO: 2; and
the V_{L} sequence set forth in SEQ ID NO: 4.

28. The monospecific anti-PD1 antibody or antigen-binding fragment thereof of claim 26, comprising:
the V_{H} sequence set forth in SEQ ID NO: 6; and
the V_{L} sequence set forth in SEQ ID NO: 8.

29. The monospecific anti-PD1 antibody or antigen-binding fragment thereof of claim 25, wherein the monospecific anti-PD1 antibody or the antigen binding fragment thereof is selected from a Fab, a Fab′, a F(ab′)₂, an Fd, an Fv, a dAb, a single-chain antibody, a humanized antibody, or a chimeric antibody.

30. The monospecific anti-PD1 antibody or antigen-binding fragment thereof of claim 25, wherein the monospecific anti-PD1 antibody or antigen-binding fragment thereof contains non-CDR regions from species other than mouse.

31. One or more nucleic acid molecules, comprising:
i) a nucleic acid sequence encoding the V_{H} of the monospecific anti-PD1 antibody or antigen-binding fragment thereof of claim 25; and
ii) a nucleic acid sequence encoding the V_{L} of the monospecific anti-PD1 antibody or antigen-binding fragment thereof of claim 25.

32. An expression construct, wherein the expression construct contains the one or more nucleic acid molecules of claim 31.

33. An expression cell line, wherein the expression cell line contains the one or more nucleic acid molecules of claim 31.

34. An expression cell line, wherein the expression cell line contains the expression construct of claim 32.

35. Hybridoma cell line LT003, wherein the hybridoma cell line has been deposited in the China Center for Typical Culture Collection (CCTCC) with the CCTCC Deposit Accession NO: C2015105.

36. A conjugate, comprising:
i) the monospecific anti-PD1 antibody or antigen-binding fragment thereof of claim 25; and
ii) a detectable marker.

37. A method of detecting the existence or levels of PD1 in a biological sample, comprising:
i) contacting the biological sample and the conjugate of claim 36; and
ii) detecting the detectable marker.

38. A pharmaceutical composition comprising the monospecific anti-PD1 antibody or antigen-binding fragment thereof of claim 25.

39. A method for treating cancer, comprising administering to a subject the monospecific anti-PD1 antibody or antigen-binding fragment thereof of claim 25.

40. The method of claim 39, wherein the cancer is melanoma, renal cancer, prostate cancer, bladder cancer, colorectal cancer, gastrointestinal cancer, liver cancer, non-small cell lung cancer, ovarian cancer, or leukemia.

41. A method for treating anemia, comprising administering to a subject the monospecific anti-PD1 antibody or the antigen-binding fragment thereof of claim 25.


SEQUENCE LISTING (16 sequences)

SEQ ID NO: 1 - DNA, 354 bp, synthetic construct
  misc_feature 1..354: note = monoclonal antibody 14C12 heavy chain variable region
  source 1..354: mol_type = other DNA
gaggtcaaactggtggagagcggcggcgggctggtgaagcccggcgggtcactgaaactgagctgcgccgcttccggcttcgcctttagctcctacgacatgtcatgggtgaggcagacccctgagaagcgcctggaatgggtcgctactatcagcggaggcgggcgatacacctactatcctgactctgtcaaagggagattcacaattagtcgggataacgccagaaatactctgtatctgcagatgtctagtctgcggtccgaggatacagctctgtactattgtgcaaaccggtacggcgaagcatggtttgcctattggggacagggcaccctggtgacagtctctgcc

SEQ ID NO: 2 - protein, 118 aa, synthetic construct
  REGION 1..118: note = monoclonal antibody 14C12 heavy chain variable region
EVKLVESGGGLVKPGGSLKLSCAASGFAFSSYDMSWVRQTPEKRLEWVATISGGGRYTYYPDSVKGRFTISRDNARNTLYLQMSSLRSEDTALYYCANRYGEAWFAYWGQGTLVTVSA

SEQ ID NO: 3 - DNA, 318 bp, synthetic construct
  misc_feature 1..318: note = monoclonal antibody 14C12 light chain variable region
  source 1..318: mol_type = other DNA
gacattaagatgacacagtccccttcctcaatgtacgctagcctgggcgagcgagtgaccttcacatgcaaagcatcccaggacatcaacacatacctgtcttggtttcagcagaagccaggcaaaagccccaagaccctgatctaccgggccaatagactggtggacggggtccccagcagattctccggatctggcagtgggcaggattactccctgaccatcagctccctggagtatgaagacatgggcatctactattgcctgcagtatgatgagttccctctgacctttggagcaggcacaaaactggaactg

SEQ ID NO: 4 - protein, 106 aa, synthetic construct
  REGION 1..106: note = monoclonal antibody 14C12 light chain variable region
DIKMTQSPSSMYASLGERVTFTCKASQDINTYLSWFQQKPGKSPKTLIYRANRLVDGVPSRFSGSGSGQDYSLTISSLEYEDMGIYYCLQYDEFPLTFGAGTKLEL

SEQ ID NO: 5 - DNA, 354 bp, synthetic construct
  misc_feature 1..354: note = monoclonal antibody 14C12H1L1 heavy chain variable region
  source 1..354: mol_type = other DNA
gaagtgcagctggtcgagtctgggggagggctggtgcagcccggcgggtcactgcgactgagctgcgcagcttccggattcgcctttagctcctacgacatgtcctgggtgcgacaggcaccaggaaagggactggattgggtcgctactatctcaggaggcgggagatacacctactatcctgacagcgtcaagggccggttcacaatctctagagataacagtaagaacaatctgtatctgcagatgaacagcctgagggctgaggacaccgcactgtactattgtgccaaccgctacggggaagcatggtttgcctattgggggcagggaaccctggtgacagtctctagt

SEQ ID NO: 6 - protein, 118 aa, synthetic construct
  REGION 1..118: note = monoclonal antibody 14C12H1L1 heavy chain variable region
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSS

SEQ ID NO: 7 - DNA, 321 bp, synthetic construct
  misc_feature 1..321: note = monoclonal antibody 14C12H1L1 light chain variable region
  source 1..321: mol_type = other DNA
gacattcagatgactcagagcccctcctccatgtccgcctctgtgggcgacagggtcaccttcacatgccgcgctagtcaggatatcaacacctacctgagctggtttcagcagaagccagggaaaagccccaagacactgatctaccgggctaatagactggtgtctggagtcccaagtcggttcagtggctcagggagcggacaggactacactctgaccatcagctccctgcagcctgaggacatggcaacctactattgcctgcagtatgatgagttcccactgacctttggcgccgggacaaaactggagctgaag

SEQ ID NO: 8 - protein, 107 aa, synthetic construct
  REGION 1..107: note = monoclonal antibody 14C12H1L1 light chain variable region
DIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELK

SEQ ID NO: 9 - protein, 8 aa, synthetic construct
  REGION 1..8: note = heavy chain variable region CDR
GFAFSSYD

SEQ ID NO: 10 - protein, 8 aa, synthetic construct
  REGION 1..8: note = heavy chain variable region CDR
ISGGGRYT

SEQ ID NO: 11 - protein, 11 aa, synthetic construct
  REGION 1..11: note = heavy chain variable region CDR
ANRYGEAWFAY

SEQ ID NO: 12 - protein, 6 aa, synthetic construct
  REGION 1..6: note = light chain variable region CDR
QDINTY

SEQ ID NO: 13 - skipped

SEQ ID NO: 14 - protein, 9 aa, synthetic construct
  REGION 1..9: note = light chain variable region CDR
LQYDEFPLT

SEQ ID NO: 15 - protein, 399 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 16 - protein, 107 aa, Homo sapiens [sequence omitted]